(19) United States
(12) Patent Application Publication
(10) Pub. No.: US 2013/0109768 A1
(43) Pub. Date: May 2, 2013

(54) PROCESSES AND SYSTEMS FOR CONVERTING SYNTHESIS GAS TO LIQUID HYDROCARBON PRODUCT

(71) Applicants: Robert J. Saxton, Pleasanton, CA (US); Gordon R. Deppe, Clayton, CA (US); Scott Oliver, Perth (AU); David W. Parham, Houston, TX (US); Kandaswamy Jothimurugesan, Hercules, CA (US); Charles L. Kibby, Benicia, CA (US); Tapan K. Das, Albany, CA (US); Christine M. Philips, Pleasant Hill, CA (US); Richard Sasson, Friendswood, TX (US); Anne Helgeson, Broadmoor, CA (US)

(72) Inventors: Robert J. Saxton, Pleasanton, CA (US); Gordon R. Deppe, Clayton, CA (US); Scott Oliver, Perth (AU); David W. Parham, Houston, TX (US); Kandaswamy Jothimurugesan, Hercules, CA (US); Charles L. Kibby, Benicia, CA (US); Tapan K. Das, Albany, CA (US); Christine M. Philips, Pleasant Hill, CA (US); Richard Sasson, Friendswood, TX (US); Anne Helgeson, Broadmoor, CA (US)

(73) Assignee: Chevron U.S.A. Inc., San Ramon, CA (US)

(21) Appl. No.: 13/285,098

(22) Filed: Oct. 31, 2011

(51) Int. Cl. C07C 1/04; B01J 19/00

(52) U.S. Cl. 518/706; 422/187; 518/715; 585/16

(57) ABSTRACT

Processes and systems are provided for converting synthesis gas containing a mixture of H₂ and CO to liquid hydrocarbon products having a cloud point less than about 15° C. The systems utilize at least one Fischer-Tropsch reactor containing hybrid Fischer-Tropsch catalyst with cooling and separation of reactor effluent following each reactor. The low cloud point indicates that the amount of wax in the hydrocarbon products is minimized relative to conventional Fischer-Tropsch conversion. Accordingly, more economical systems can be built and operated because equipment associated with wax removal or wax treatment can be reduced or eliminated.

### BACKGROUND

The present disclosure relates to processes and systems for converting synthesis gas to liquid hydrocarbon products such as fuels.

High quality fuels remain in high demand. Fischer-Tropsch synthesis, which involves the production of hydrocarbons by the catalyzed reaction of mixtures of carbon monoxide (CO) and hydrogen (H₂), also referred to as synthesis gas or syngas, can convert carbon-based materials, such as natural gas, into liquid fuels and high-value chemicals. Fischer-Tropsch synthesis is one of the more attractive, direct and environmentally acceptable paths to high quality transportation fuels derived from natural gas. The Fischer-Tropsch process can produce a wide variety of materials depending on catalyst and process conditions. Fischer-Tropsch catalysts are based on group VIII metals such as, for example, iron, cobalt, nickel and ruthenium. For example, cobalt and ruthenium make primarily paraffinic products, cobalt tending towards a heavier product slate, e.g., containing C₂₀₋, while ruthenium tends to produce more distillate type paraffins, e.g., C₅-C₂₀. Processes using such catalyst are generally governed by the Anderson-Schulz-Flory (ASF) polymerization kinetics. In general the product distribution of hydrocarbons formed during the Fischer-Tropsch process can be expressed as:

Wₙ/n=(1−α)²αⁿ⁻¹

where Wₙ is the weight fraction of hydrocarbon molecules containing n carbon atoms, and α is the chain growth probability for a given catalyst and process conditions.

Typically, the high proportion of normal paraffins in the product must be converted into more useable products, such as transportation fuels. This conversion is accomplished primarily by hydrogen treatments involving hydrotreating, hydroisomerization, and hydrocracking The very long-chained hydrocarbons, i.e., the C₂₁₊ fraction, are waxes which are solid at room temperature. Wax products provide poor cold flow properties making such products difficult or impossible to use where cold flow properties are vital, e.g., in lubes, gasoline fuels, diesel fuels, jet fuels. Therefore, for production of liquid transportation fuels it may be necessary to crack some of the Fischer-Tropsch wax products. Typically, this will involve further commercial processes such as hydrocracking and/or hydrotreating. These additional processes involve complex and expensive hardware that adds greatly to the capital expenditures and operational expenses in refining. In areas such as offshore platforms, hydrocracking is undesirable due to space and safety limitations.

Hybrid Fischer-Tropsch catalyst systems which further include an acidic component, such as a zeolite, have been developed which are capable of limiting product chain growth in the Fischer-Tropsch reaction to a desired product distribution.

Known commercial processes for converting synthesis gas to liquid hydrocarbon products utilizing Fischer-Tropsch processes produce an effluent which contains a solid wax fraction along with C₁₋₄ light gas, C₅₊ liquid products and water. Upon leaving the reactor, the wax fraction must be separated from the effluent before the light gas, liquid products and water can be separated from one another and further processed. To remove the wax fraction, a heated separation step is generally necessary to keep the wax in a liquid state. In some cases, significant amounts of wax must be removed from the effluent, in some cases up to 50% by weight based on the total effluent. Once the wax is separated, degassing of the wax in a flash separation step is also required in order to remove methane from the wax. In slurry bed operations, wax must be separated from the catalyst, following which the wax is filtered.

The presence of a solid wax fraction in the effluent is problematic in several ways. For one, the wax fraction can result in plugging of pumps and orifices of other fluid handling equipment or inhibiting flow of products through conduits. Fischer-Tropsch wax typically congeals and stops flowing at about 80° C., so keeping conduits heated with tracing or jacketing is generally required at any ambient temperature. At ambient temps below 0° C., problems with steam tracing often occur anywhere such that condensate within the steam tracing freezes which stops the flow of fresh steam. This leads to cold spots in conduits and wax freezing up. Fischer-Tropsch wax generally has a high congealing point temperature with a narrow range such that it hardens very quickly on surfaces at cold spots within lines and equipment. When flow disturbances occur, operations must be halted to locate and remediate the plugging. Flow assurance dictates installing expensive heating systems such as oil- or steam-jacketing or elaborate steam tracing; these systems add both to the capital and operating expenses of the facility.

Wax also can result in emulsions wherein small particles of wax are dispersed in a flowable liquid. Emulsions can form within the system in the liquid effluent as a result of wax carry-over with the reaction products, including reaction water, or in the light overheads coming from a Fischer-Tropsch reactor as a result of small amounts of wax becoming entrained in the overheads. Reactor overhead products and reaction water are cooled so they will condense into liquids for recovery. In the process, waxes have a tendency to freeze inside the condenser tubes. Initially, the wax deposits on the surface of the condenser tubes, resulting in losses in heat transfer efficiency, so recoverable hydrocarbons are not condensed and captured. If not remediated early, then the wax deposits grow so large that they restrict flow, leading to pressure control problems inside the reactor, resulting in a potential uncontrolled shutdown and/or pressure-relief valve activating. Equipment must then be taken off-line to remove the wax deposits inside the overhead condensers and separators, either manually using a water cannon or chemically by circulating a hot solvent through the equipment.

In order to prevent the formation of emulsions, de-emulsifiers or anti-foaming agents are commonly added to equipment such as separators. A problem with Fischer-Tropsch wax is that the characteristics of the emulsions change over time, so that it is difficult to identify a solution that will prevent the formation of emulsions consistently. The emulsions change because the wax produced changes as the Fischer-Tropsch catalyst ages and degrades.

Emulsions in the water/hydrocarbon separators in a given Fischer-Tropsch system make level control at the water/hydrocarbon interface very difficult. In situations where the water/hydrocarbon interface is not controlled with anti-foaming agents, one of two things will happen. For one, hydrocarbons can get into the wastewater system, causing wax freezing in the wastewater system and hydrocarbon spills on the ground. For another, water can get into the hydrocarbon systems, which can damage catalysts in the hydrocracking/hydrotreating equipment downstream of the water/wax separator and cause a variety of operational upsets.

The presence of the wax fraction in the effluent can also lead to problems within the Fischer-Tropsch reactor. In fixed bed operations, wax can coat and plug the pores of the catalyst, thereby reducing the effective life of the catalyst. Catalyst-wax separation in a slurry bed reactor is difficult since the catalyst particles are brittle, thus requiring gentle handling to avoid fracturing.

Liquid hydrocarbon product containing wax can be blended with crude oil, however, wax containing product can generally only be added at a rate of 2-3% by volume of total crude in order to avoid problems associated with the presence of wax. For instance, blending Fischer-Tropsch product containing wax into crude oil can increase the pour point of the crude oil above 60° C. Furthermore, the wax precipitation temperature or wax appearance temperature of crude oil blends is lower than crude oil not containing Fischer-Tropsch product. When transporting crude oil blends containing wax, the temperature must be maintained above the cloud point in order to prevent wax deposition in cargo tanks and conduits used for loading and unloading. Pour point depressants have been used in attempts to mitigate this problem.

There is a need for economical processes and systems capable of utilizing a hybrid Fischer-Tropsch catalyst system to produce a liquid hydrocarbon product blendable with a variety of crude oils and having a cloud point below about 15° C. without the need for hydrocracking, thus avoiding the aforementioned difficulties.

### SUMMARY

According to one embodiment, a system is provided for producing liquid hydrocarbons which includes at least one reactor wherein a first synthesis gas feed, in the presence of a catalyst comprising a synthesis gas conversion component and an acidic component, is converted into an effluent comprising gaseous components, water and liquid hydrocarbons having a cloud point less than about 15° C. as determined by ASTM D 2500-09 and a separator for separating the effluent into the gaseous components, the water and the liquid hydrocarbons. The effluent is provided to the separator without the removal of a solid wax phase.

According to another embodiment, a process is provided for converting synthesis gas to a liquid hydrocarbon product. A synthesis gas feed comprising hydrogen and carbon monoxide having a H₂/CO ratio between about 0.5 and about 2.5 is contacted with a catalyst comprising a synthesis gas conversion component and an acidic component in a reactor at a temperature between about 160° C. and about 350° C., a pressure between about 1 and about 100 atmospheres, and a gaseous hourly space velocity less than 20,000 volumes of gas per volume of catalyst per hour to produce an effluent. The effluent is separated in a separator at a temperature of at least about 0° C. into a gaseous phase, an aqueous phase and a liquid hydrocarbon phase having a cloud point less than about 15° C. as determined by ASTM D 2500-09. The effluent is provided to the separator without the removal of a solid wax phase.

### DESCRIPTION OF THE DRAWINGS

These and other objects, features and advantages of the present invention will become better understood with regard to the following description, appended claims and accompanying drawings where:

FIG. 1 is a block diagram illustrating a process for converting synthesis gas to liquid fuel according to one embodiment.

FIG. 2 is a block diagram illustrating a process for converting synthesis gas to liquid fuel according to another embodiment.

FIG. 3 is a block diagram illustrating a process for converting synthesis gas to liquid fuel according to another embodiment.

### DETAILED DESCRIPTION

Disclosed herein are a process and a system for converting synthesis gas comprising hydrogen and carbon monoxide, also referred to as syngas, to a liquid hydrocarbon product suitable for use as a fuel.

In one process embodiment, a syngas feed is contacted with a catalyst having a synthesis gas conversion component and an acidic component in a first reactor. The reaction in the first reactor occurs at a temperature between about 160° C. and about 350° C., even between about 200° C. and about 250° C. The pressure in the first reactor is between about 1 and about 100 atmospheres, even between about 10 atmospheres and about 30 atmospheres. The gaseous hourly space velocity of the reaction is less than about 20,000 volumes of gas per volume of catalyst per hour. The syngas has a H₂/CO ratio between about 0.5 and about 2.5, even between about 1.0 and about 2.0, and even between about 1.2 and about 1.7. An effluent stream is produced which contains water and hydrocarbons as well as gaseous components.

Unlike conventional Fischer Tropsch processes in which the reactor effluent must be separated in a high-temperature separation to remove a solid wax fraction prior to further phase separation of the effluent, in the present process, no such wax separation step is required. According to the present process, upon leaving the reactor, the effluent is cooled, thereby forming a first vapor phase comprising C₁₋₄ and CO₂, a liquid aqueous phase and a first liquid hydrocarbons product stream comprising C₅₊ hydrocarbons and having a cloud point less than about 15° C. as determined by ASTM D 2500-09. The effluent can be cooled by one or more known suitable means. For instance, the effluent can be cooled in a cross exchanger with the reactor feed, in a feed-effluent exchanger. The effluent can be further cooled in one or more coolers utilizing air and/or water. The lower the temperature to which the effluent is cooled, the better the liquid hydrocarbons recovery.

The gas and liquid phases are then separated in a three-phase gas/hydrocarbon liquid/water separation unit. The effluent stream can be condensed without forming a solid wax phase which would conventionally require wax separation prior to gas/hydrocarbon liquid/water separations. The aqueous phase, the liquid hydrocarbons product stream and the vapor phase of the effluent are therefore separated in the three-phase separation unit without prior wax removal. The separator unit can operate at a temperature of at least about 0° C., even between about 0° C. and about 100° C., even between about 0° C. and about 66° C.

The liquid hydrocarbon stream can optionally be sent to a product accumulator. Alternatively, the liquid hydrocarbon stream can optionally be sent to a hydroprocessing step such as mild hydrogenation, also referred to herein as hydrofinishing, to convert olefins to paraffins or linear alkanes. Hydrofinishing is typically conducted at temperatures ranging from about 190° C. to about 340° C. at pressures from about 400 psig to about 3000 psig (2.76 to 20.7 MPa gauge) at space velocities (LHSV) between about 0.1 and 20 and a hydrogen recycle rate of about 400 to 1500 SCF/bbl (0.071 to 0.27 SCM/liter). Suitable hydrofinishing catalysts include noble metals from Group VIIIA (according to the 1975 rules of the International Union of Pure and Applied Chemistry), such as platinum or palladium on an alumina or siliceous matrix, and unsulfided Group VIIIA and Group VIB, such as nickel-molybdenum or nickel-tin on an alumina or siliceous matrix. U.S. Pat. No. 3,852,207 describes a suitable noble metal catalyst and mild conditions. Other suitable catalysts are described, for example, in U.S. Pat. Nos. 4,157,294 and 3,904,513.

Because of the extremely low level of C₂₁₊ fraction in the liquid hydrocarbon stream, it may be transported at ambient temperature through lines, pumped to higher elevation, stored in a tank, returned to the first reactor and/or sent to a second reactor for further reaction without the risk of wax freezing within hardware such as lines, pumps, valves, tanks and the like. Furthermore, it is not required to heat or preheat such hardware.

In one exemplary embodiment, the first vapor phase contains primarily hydrogen, CO, CO₂ and C₁₋₄ hydrocarbons. The H₂/CO ratio in the first vapor phase generally is between about 0 and about 1.5:1, more in particular between about 0.1 and about 0.9:1, still more in particular between about 0.3 and about 0.9:1. The first vapor phase may also comprise other components such as inert gases like nitrogen. The first vapor phase is next combined with additional makeup hydrogen and sent to a second reactor. Makeup hydrogen is added to the first vapor phase to form a second synthesis gas feed having a H₂/CO ratio between about 0.5 and about 2.5, even between about 1.0 and about 2.0, even between about 1.2 and about 1.7. The H₂/CO ratio of the makeup hydrogen generally is at least about 2:1.

In the second reactor, the second synthesis gas feed is contacted with a second catalyst comprising a synthesis gas conversion component and an acidic component at a temperature, a pressure, and a gaseous hourly space velocity within the ranges described above with respect to the first reactor to produce a second effluent stream. Again, the second effluent stream is cooled to form a second vapor phase comprising C₁₋₄ and CO₂, a second aqueous phase and a second liquid hydrocarbons product stream containing C₅₊ hydrocarbons and having a cloud point less than about 15° C. as determined by ASTM D 2500-09. The phases are separated in a second three-phase (gas/hydrocarbon liquid/water) separation unit. Those skilled in the art will appreciate that a series of separators may be used rather than a single three-phase separator. Multiple separation units allow greater process flexibility, since gas can recycled from the first separator, second separator or both. Advantageously, the H₂/CO ratio of the synthesis gas feed entering the first and/or second reactor can be adjusted by recycling gas from the second three-phase separation unit. The liquid hydrocarbons product streams from the first and second reactors are combined by any convenient means to form a final liquid hydrocarbon product. The liquid hydrocarbon product streams can optionally be combined in a product accumulator. Alternatively, the liquid hydrocarbon stream can be sent to an optional hydrofinishing step, as described above.

According to one embodiment, the final liquid hydrocarbon product includes:
i. 0 to 20 wt % CH₄; and
ii. 0 to 5 wt % C₂₁₊ normal paraffins.

Preferably, the final product further includes:
iii. 0 to 30 wt % C₂-C₄; and
iv. 50 to 95 wt % C₅₊.

The liquid hydrocarbon product has a cloud point as determined by ASTM D 2500-09 of about 15° C. or less, even about 10° C. or less, even about 5° C. or less, and even as low as about 2° C. Cloud point refers to the temperature below which wax in a liquid hydrocarbon product forms a cloudy appearance as the wax forms an emulsion with the liquid phase of the product. Cloud point indicates the tendency of the product to plug pumps, filters or small orifices at cold operating temperatures. Note that a 6° C. cloud point is typical for a Number 2 diesel.

The system may further include recycle of tail gas from any or all of the separation units back to the first reactor, the second reactor or to both reactors. In the case that tail gas is recycled to the first reactor, the ratio between fresh syngas and recycle gaseous effluent can be between about 0.1 and about 10:1, even between about 0.2 and about 5:1, even between about 0.3 and about 3:1. It is noted that ratios provided herein between gas streams are volume ratios, and H₂/CO ratios are molar ratios.

In one embodiment, the volume of catalyst in the second reactor is lower than the volume of catalyst in the first reactor. A significant reduction in catalyst volume can be realized using the first and second reactors in series versus a single reactor in order to achieve equivalent percent CO conversion, since the reaction rate is maintained at a higher level in the first and second reactors versus a single reactor. This is because the partial pressure of the reactants (H₂ and CO) entering the second reactor is higher than the partial pressure of the reactants in the lower portion of a single reactor.

The present process and system result in high overall CO conversion, up to about 99 mol %, accompanied by a high selectivity to C₅₊ hydrocarbon products. The CO conversion can easily be changed by increasing or decreasing the reactor temperature and/or the reaction pressure, and by adapting the recycle conditions. Even with no recycle of tail gas, the present process can achieve about 85 mol % CO conversion. Suitably the conversion of CO that is fed to the first and second reactor is between about 1 and about 80 mol %, preferably between about 20 and about 60 mol %. The CO conversion can easily be changed by increasing or decreasing the reactor temperature and/or the reaction pressure, and by adapting the recycle conditions.

The present process can be adapted to various syngas compositions, including syngas with a relatively low H₂/CO ratio, which allows the use of the same design in different operations. The syngas may, for example, be obtained from natural gas, but also from peat, coal, biomass, or other hydrocarbon fractions by processes like gasification, autothermal reforming, catalytic or non-catalytic partial oxidation.

If so desired, the process may include third- and further-stage reactors, the effluent of which may be fed to third and fourth three-phase separation units. The third- and further-stage reactors may optionally be fed with additional makeup hydrogen.

Numerous types of reactor systems have been developed for carrying out the Fischer-Tropsch reaction. For example, Fischer-Tropsch reactor systems include fixed bed reactors, especially multitubular fixed bed reactors, fluidized bed reactors, such as entrained fluidized bed reactors and fixed fluidized bed reactors, and slurry bed reactors such as three-phase slurry bubble columns and ebullated bed reactors. The present invention is applicable to all types of reactor systems. The reactors each have an inlet for receiving synthesis gas and an outlet for discharging an effluent stream.

In the reactor, the syngas is partially converted into hydrocarbon products under conversion conditions, accompanied by the formation of water. This is done by contacting the syngas with a catalyst. Suitable catalysts for minimal formation of C₂₁₊ fraction have recently been developed and will be discussed below.

In some embodiments, the first and/or the second stage Fischer-Tropsch reactor can include a set of two or more subreactors operated in parallel. Therefore, where in the present specification reference is made to, for example, the first stage Fischer-Tropsch reactor, this also encompasses a set of two or more first stage Fischer-Tropsch reactors operated in parallel. In one embodiment of the present process, a first-stage reactor is used which comprises at least two subreactors and a second-stage reactor is used which comprises at least two subreactors, wherein fresh syngas feed lines, additional makeup hydrogen feed lines, and tail gas recycle feed lines to both the first stage subreactors and the second stage subreactors. In operation, each reactor will be provided with either fresh syngas or additional makeup hydrogen. This allows the selection of which reactor will operate as first stage reactor, and which reactor will operate as second stage reactor.

The syngas conversion or Fischer-Tropsch component of the catalyst comprises a Group VIII metal component, preferably cobalt, iron and/or ruthenium. References to the Periodic Table and groups thereof used herein refer to the IUPAC version of the Periodic Table of Elements described in the 68th Edition of the Handbook of Chemistry and Physics (CPC Press). The catalyst further comprises a catalyst carrier or support. The catalyst carrier is preferably porous, such as a porous inorganic refractory oxide, preferably alumina, silica, titania, zirconia or combinations thereof. The optimum amount of catalytically active metal present on the carrier depends inter alia on the specific catalytically active metal. Typically, the amount of cobalt present in the catalyst may range from about 1 to about 100 parts by weight per 100 parts by weight of carrier material, preferably from about 10 to about 50 parts by weight per 100 parts by weight of carrier material.

The catalytically active Fischer-Tropsch component may be present in the catalyst together with one or more metal promoters or co-catalysts. The promoters may be present as metals or as metal oxide, depending upon the particular promoter concerned. Suitable promoters include metals or oxides of metals from Groups IA, IB, IVB, VB, VIB and/or VIIB of the Periodic Table, lanthanides and/or the actinides or oxides of the lanthanides and/or the actinides. As an alternative or in addition to the metal oxide promoter, the catalyst may comprise a metal promoter selected from Groups VIIB and/or VIII of the Periodic Table.

The acidic component of the catalyst is an acid catalyst material such as amorphous silica-alumina or tungstated zirconia or a zeolitic or non-zeolitic crystalline molecular sieve. Examples of suitable hydrocracking molecular sieves include zeolite Y, zeolite X and the so called ultra stable zeolite Y and high structural silica:alumina ratio zeolite Y such as for example described in U.S. Pat. Nos. 4,401,556, 4,820,402 and 5,059,567, herein incorporated by reference. Small crystal size zeolite Y, such as described in U.S. Pat. No. 5,073,530, herein incorporated by reference, can also be used. Other zeolites which show utility as cracking catalysts include those designated as SSZ-13, SSZ-33, SSZ-46, SSZ-53, SSZ-55, SSZ-57, SSZ-58, SSZ-59, SSZ-64, ZSM-5, ZSM-11, ZSM-12, ZSM-23, H—Y, beta, mordenite, SSZ-74, ZSM-48, TON type zeolites, ferrierite, SSZ-60 and SSZ-70. Non-zeolitic molecular sieves which can be used include, for example silicoaluminophosphates (SAPO), ferroaluminophosphate, titanium aluminophosphate and the various ELAPO molecular sieves described in U.S. Pat. No. 4,913,799 and the references cited therein. Details regarding the preparation of various non-zeolite molecular sieves can be found in U.S. Pat. No. 5,114,563 (SAPO); U.S. Pat. No. 4,913,799 and the various references cited in U.S. Pat. No. 4,913,799, hereby incorporated by reference in their entirety. Mesoporous molecular sieves can also be included, for example the M41S family of materials (J. Am. Chem. Soc. 1992, 114, 10834-10843), MCM-41 (U.S. Pat. Nos. 5,246,689, 5,198,203, 5,334,368), and MCM48 (Kresge et al., Nature 359 (1992) 710).

The amount of acidic component used in the catalyst can be suitably varied to obtain the desired product. If the amount of acidic component is too low, there will be insufficient cracking to remove all of the wax; whereas if too much acidic component is used, there will be too much cracking and the resulting product may be too light.

In one embodiment, the catalyst comprises synthesis gas conversion component and acidic component disposed on integral particles such as catalysts described in U.S. Patent Publication No. 2010/0160464 A1, herein incorporated by reference in its entirety.

In an alternate embodiment, the catalyst components are arranged in a “stacked bed” configuration wherein a catalyst bed of synthesis gas conversion component in a reactor is followed by a downstream bed of the acidic component, as described in U.S. Patent Publication No. 2010/0312030 A1, herein incorporated by reference in its entirety.

In yet another embodiment, the synthesis gas conversion component and the acidic component are disposed on discrete, separate particles which are mixed together to form a single catalyst bed. Such catalysts are described in U.S. Pat. No. 7,825,164, herein incorporated by reference in its entirety.

In one embodiment, the first catalyst is free of acidic component, such that the first effluent stream contains a wax fraction of up to 30 wt % C₂₁₊ normal paraffins which is subsequently cracked by a sufficient amount of acidic component in the second reactor.

In one embodiment, the process further includes hydrogenating the liquid C₅₊ hydrocarbon product in order to saturate the olefins present.

An advantage of the present process is that the liquid C₅₊ hydrocarbon product need not be further hydrocracked or hydroisomerized in order to arrive at a desirable liquid, wax free product composition.

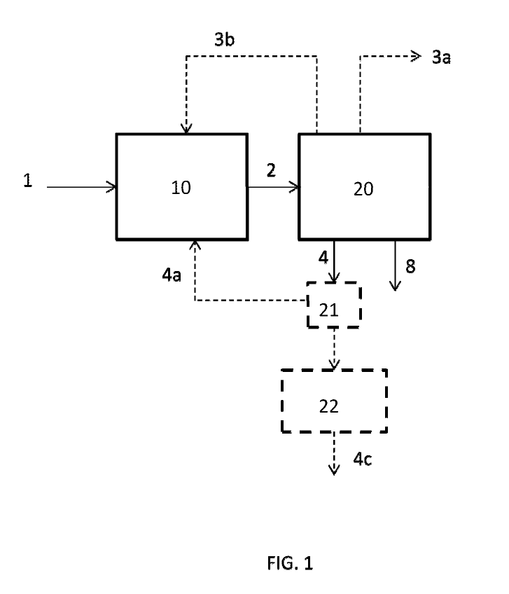

One embodiment of the present disclosure is illustrated in FIG. 1. Fresh syngas, comprising CO and H₂, provided through feed line 1, is combined with optional recycle (not shown) of tail gas 3 to form a feed which is fed to a Fischer-Tropsch reactor 10. The effluent 2 from the Fischer-Tropsch reactor 10 is fed to a three phase (i.e., gas/hydrocarbon liquid/water) separation unit 20 without the separation of a solid phase. In separation unit 20, effluent from the reactor is separated to form liquid hydrocarbon product stream 4, aqueous stream 8 and vapor phase or tail gas 3. Tail gas stream 3 is optionally compressed (not shown) and recycled to the reactor, with or without the addition of makeup hydrogen. The separation unit 20 can be operated at a temperature of at least about 0° C.

In one embodiment, all or a portion of liquid hydrocarbon product stream 4 can be recycled to Fischer-Tropsch reactor 10 as liquid stream 4a in order to obtain a desired product distribution. In order to collect sufficient liquid to recycle at a reasonable rate, a reservoir 21 can be used. Liquid stream 4a is pumped from the reservoir to a liquid distributor (not shown) within the reactor 10 by a pump (not shown). Recycle of a portion of liquid hydrocarbon product stream 4 can occur continuously. Alternatively, recycle of all or a portion of liquid hydrocarbon product stream 4 can occur intermittently in order to obtain a desired product distribution.

In one embodiment, the liquid hydrocarbon product stream 4 can be sent to a hydrofinishing reactor 22 to form a hydrofinished liquid hydrocarbon product stream 4c.

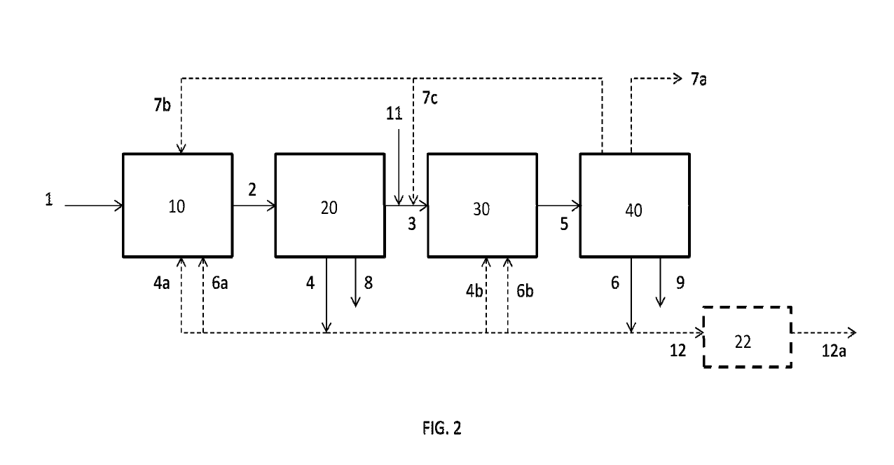

Another embodiment of the present disclosure is illustrated in FIG. 2. Fresh syngas, comprising CO and H₂, provided through feed line 1, is combined with optional recycle of tail gas 7b to form a combined feed, which is fed to a first Fischer-Tropsch reactor 10. The effluent 2 from the first Fischer-Tropsch reactor 10 is fed to separation unit 20. In separation unit 20, effluent from the first Fischer-Tropsch reactor is separated to form liquid hydrocarbon product stream 4, first aqueous stream 8 and first vapor phase 3. The first vapor phase 3 is combined with makeup hydrogen 11 and fed to a second Fischer-Tropsch reactor 30. The second effluent 5 from the second Fischer-Tropsch reactor 30 is fed to separation unit 40 where the second effluent is separated to form liquid hydrocarbon product stream 6, second aqueous stream 9 and tail gas. The tail gas can be removed as stream 7a, compressed and recycled to the first Fischer-Tropsch reactor 10 as stream 7b and/or compressed and recycled to the second Fischer-Tropsch reactor 30 as stream 7c.

Liquid hydrocarbon product streams 4 and/or 6 can optionally be sent to the Fischer-Tropsch reactors 10 and/or 30 to obtain a desired product distribution. As shown, all or a portion of liquid product stream 4 can be recycled to reactor 10 as liquid stream 4a, as described above with regards to FIG. 1. Similarly, all or a portion of liquid product stream 4 can be sent to reactor or 30 as liquid stream 4b. Similarly, all or a portion of liquid product stream 6 can be recycled to reactor 10 and/or reactor 30 as streams 6a and/or 6b, respectively. Reservoirs for collecting liquid product streams 4 and 6 and pumps for sending liquid from the reservoirs to the reactors are not shown.

Product streams 4 and 6 can be combined to yield the accumulated product 12 which has a cloud point less than about 15° C. as determined by ASTM D 2500-09. Combined liquid hydrocarbon product stream 12 is optionally sent to a hydrofinishing reactor 22 to form hydrofinished liquid hydrocarbon product stream 12a. Lines carrying effluent 2 between first reactor 10 and separation unit 20, as well as valve 25 and pump 26 need not be provided with a source of heat. Similarly, lines carrying effluent 5 need not be provided with a source of heat.

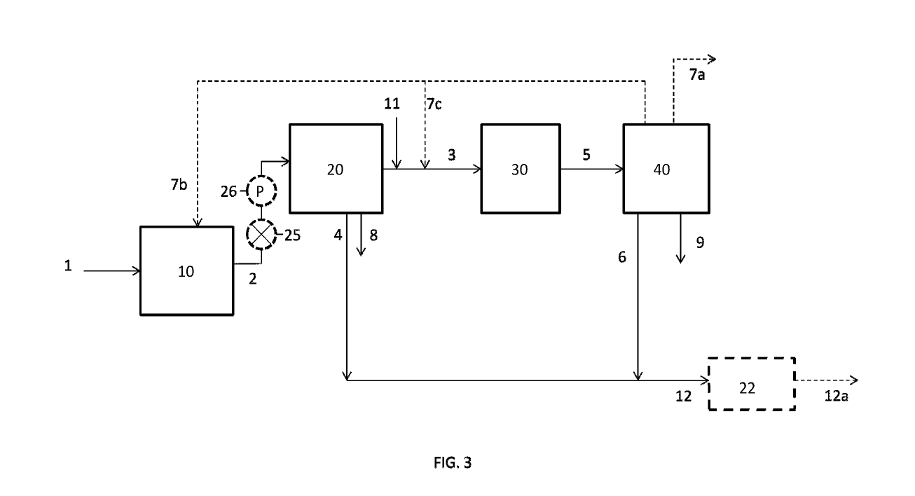

Another embodiment of the present disclosure, similar to the system of FIG. 2, is illustrated in FIG. 3. In this embodiment, the separation unit 20 and the second Fischer-Tropsch reactor 30 are located at an elevated height relative to the first reactor 10. By contrast, conventional Fischer-Tropsch systems do not include pumping synthesis products to a higher elevation because of the wax content. Optional valve 25 is provided for controlling the flow of effluent 2 and optional pump 26 is provided for pumping effluent 2 from the first reactor 10 to the separation unit 20.

In each of the systems illustrated in FIGS. 1-3, the lines for removing the first and second aqueous streams 8 and 9 need not be formed of a highly corrosion resistant alloy. Carbon steel pipe may be used rather than corrosion resistance mixtures of various metals such as stainless steel, chrome, nickel, iron, copper, cobalt, molybdenum, tungsten and/or titanium.

### EXAMPLES

### Example 1

A hybrid Fischer-Tropsch catalyst of composition 7.5% Co/0.19% Ru/ZSM-5/Al₂O₃ was prepared as described in U.S. Patent Publication No. 2010/0160464A, herein incorporated by reference in its entirety. The catalyst was placed in a 9.52 mm reactor tube. The catalyst was subjected to activation and a Fischer-Tropsch synthesis run as generally described in U.S. Pat. No. 7,943,674, herein incorporated by reference in its entirety. The reaction was run at a temperature of 225° C. and a pressure of 132 psig, using a synthesis gas feed having a H₂/CO ratio of 1.6, with no recycle of tail gas.

Total Acid Number (TAN) was measured according to ASTM D664-09 and reported in milligrams KOH per gram of sample for samples of the liquid hydrocarbon and aqueous phases produced in the reaction. The results are given as follows:

| | TAN (mgKOH/g) |
|---|---|
| Liquid hydrocarbon phase | 0.46 |
| Aqueous phase | 0.49 |

Reactor wastewater treatment guidelines generally specify a chemical oxygen demand (COD) requirement, which is the amount of oxygen required to oxidize organic compounds completely to CO₂ and H₂O, according to the following formula:

CₙHₐO_{b}N_{c}+(n+a/4−b/2−3/4c)*O₂→nCO2+(a/2−3/2c)*H₂O+cNH₃

The COD on a molar basis is calculated as the term preceding the oxygen in the above formula, i.e., (n+a/4−b/2−3/4c).

A wastewater sample of water produced by the reaction of approximately 470 ml was acidified to pH of 1 and extracted with 50 ml, 10 ml and 20 ml of methylene chloride. A 3 ml aliquot of the combined methylene chloride extract was evaporated under a stream of nitrogen, yielding 1.2 mg of dried extract. This extract was analyzed by infrared (IR) and Gas chromatography-mass spectroscopy (GC/MS).

The evaporated methylene chloride extract analyzed by IR clearly showed the presence of acids and possibly a small amount of alcohol. The GC/MS analysis also showed peaks that were consistent with the presence of acids containing between 5 to 10 carbons. Assuming that all of the 1.2 mg of evaporated extract obtained by evaporating 3 ml of methylene chloride contains acids, the concentration of acids in the water sample would be approximately 68 ppm. Based on the 68 ppm result for acid concentration and the C₅₋₁₀ distribution of organic acids based on GC/MS results, the COD was determined to be approximately 150 mg/l. Such water would therefore require minimal treatment prior to disposal.

By contrast, reaction wastewater from a conventional Fischer-Tropsch process using a Fischer-Tropsch catalyst of cobalt supported on alumina and has approximately 3.5% by weight of oxygenated organics, corresponding to a COD of approximately 54,000 mg/l.

### Example 2

The Fischer-Tropsch synthesis reaction of Example 1 was repeated at the same conditions with the exception of recycle of tail gas at a recycle ratio of 2.

A wastewater sample produced by the reaction was submitted for analysis by GC/MS to look for the presence of hydrocarbons, acids and alcohols. A water sample of approximately 190 ml was acidified to a pH of 1 with concentrated hydrochloric acid and extracted with 50 ml, 20 ml and 10 ml of methylene chloride. The combined extract was dried with sodium sulfate. A 10 ml aliquot of the combined extracts was evaporated to approximately 1 ml and analyzed by GC/MS. A 6 ml aliquot of the combined methylene chloride extract was evaporated to dryness under a stream of nitrogen, yielding 1.2 mg of dried extract The 1.2 mg of extract was analyzed by GC/MS. The remaining methylene chloride extract was evaporated and analyzed by IR.

The GC/MS trace of the evaporated 10 ml aliquot, showed the presence of normal and branched hydrocarbons ranging from about C₁₂₋₂₂. Olefins also appear to be present but their low concentration makes it difficult to verify from the mass spectral data. Acids containing between 5 and 9 carbons were indicated. The IR of the evaporated methylene chloride extract showed a strong acid peak at 1703.592 and a weak OH peak indicating very little alcohol. If all of the 1.2 mg of evaporated extract obtained by evaporating 6 ml of methylene chloride consisted of acids, the concentration of acids in the water sample would be approximately 126 ppm. However, the 1.2 mg of extract also contains branched and normal alkanes so the acid concentration is actually less than 126 ppm. The COD of the wastewater was calculated to be approximately 280 mg/l. Such water would therefore require minimal treatment prior to disposal.

### Example 3

A hybrid Fischer-Tropsch catalyst containing 7.5% Co-0.19% Ru on 1/16″ alumina-bound ZSM-12 extrudates was prepared in described in the Examples disclosed in U.S. Pat. No. 7,943,674. Properties of the extrudate and catalyst are set forth in Table 1.

TABLE 1

| | Micro- | External | BET | | Average |
|---|---|---|---|---|---|
| | pore | Surface | Surface | Metal | Particle |
| Catalyst | Area, | Area, | Area, | Disper- | Diameter, |
| Composition | m²/g | m²/g | m²/g | sion, % | nm |
| 80% ZSM-12 + | 167 | 98 | 264 | na | na |
| 20%Al₂O₃ | | | | | |
| 7.5%Co—0.19Ru/ | 55 | 61 | 115 | 15.2 | 6.5 |
| (80% ZSM-12 + | | | | | |
| 20% Al₂O₃) | | | | | |

Ten grams of the catalyst was charged to a glass tube reactor and activated as disclosed in U.S. Pat. No. 7,943,674. The catalyst was subjected to a synthesis run in which the catalyst was contacted with hydrogen and carbon monoxide in ratio of 2.0 at a temperature of 220° C. with a total pressure of 20 atm and a space velocity of 1200 mL of gas (0° C., 1 atm) per gram of catalyst per hour. After the separation of water from the liquid product, the liquid hydrocarbon product was passed through a ¼ inch (0.63 cm) diameter tube reactor loaded with 2 g of NiMo hydrofinishing catalyst (available from Albemarle Corporation) at a temperature of 220° C., a pressure of 15 atm and a weight hourly space velocity of 1. The bromine number of the hydrofinished product was determined by test ASTM D 1159 to be less than 1, indicating complete saturation of all olefins.

The processes and systems of the present disclosure provide numerous advantages as compared with conventional Fischer-Tropsch processes and systems. The need to handle and remove a wax fraction following the reactor is reduced. This simplifies the system and reduces energy usage by eliminating a heated wax separation unit. Since there is no solid wax fraction in the reactor effluent, plugging of pumps and orifices of fluid handling equipment or inhibition of the flow of products through system conduits can be minimized. As a result, costly system shutdowns to remediate such problems can be avoided. Furthermore, costly measures such as expensive heat tracing designed to prevent such problems can be avoided. One advantage of the present process is that prior to starting up the Fischer-Tropsch reaction, the reactor and lines coming from the reactor need not be preheated as is typically required to avoid freezing of wax.

In the processes disclosed herein, the formation of emulsions of small particles of wax can be reduced in the reactor effluent, including the reaction water, or in the light overheads coming from the top of the reactor. This can reduce the potential for plugging at cold spots in the system. This can also reduce the need for de-emulsifiers or anti-foaming agents in system components such as separators.

The systems disclosed herein have the advantage of improved interface level control in the water separation unit, such that separation of clean water from reaction water may be easier. This may result in less waste-water remediation required.

Another advantage of the present systems is lower oxygenate content in the produced hydrocarbons and produced water. Fischer-Tropsch reactions produce hydrocarbons and oxygenates including alcohols, acids and water. The acidic component of the hybrid Fischer-Tropsch catalysts disclosed herein efficiently catalyzes the dehydration of the oxygenate products to yield hydrocarbons and water. Thus, products according to the present disclosure contain lower levels of oxygenates than conventional Fischer-Tropsch products. One consequence of this is reduced acid content in the reaction water (also referred to as produced water), which can result in cost savings in terms of equipment and reaction water treatment. Acid content in the reaction water produced by the present process can have a Total Acid Number (TAN) less than about 0.5, which is less than about 10% of the TAN of conventional Fischer-Tropsch produced water. As a result, there may be a reduced need for neutralizing the pH of the produced water using corrosion inhibiting buffers or caustic agents. There may also be a reduced need for expensive corrosion resistant alloys in hardware such as lines and tanks As a result of lower oxygenate content, there is also reduced acid content in the liquid hydrocarbon product, resulting in a less corrosive product. As result, corrosion mitigation such as expensive alloys in the product handling hardware can be avoided.

The processes and systems of the present disclosure may have lower risk of fire than conventional Fischer-Tropsch systems since there is reduced flammable wax fraction in the system which could accumulate on surfaces creating a potential fire hazard.

Another advantage of the present processes and systems is the reduced tendency for catalyst deactivation in fixed bed operations, and therefore longer catalyst life, as compared with conventional Fischer-Tropsch operations, because of the absence of a wax fraction in the reaction products.

Liquid hydrocarbon product produced by the present processes can be blended with crude oil. It may be possible to blend the product produced by processes disclosed herein at higher amounts by volume without encountering typical problems associated with blending a conventional Fischer-Tropsch wax containing product with crude oil.

Systems according to the present disclosure can advantageously be deployed in a number of ways. In some embodiments, such a system can be located at a hydrocarbon production facility in which oil and gas are extracted from an oil-producing well. The present system can be employed to convert syngas obtained from the natural gas associated with oil production, also referred to as associated gas, to valuable liquid hydrocarbons. This can be particularly advantageous at remote landlocked locations and offshore production facilities, thus avoiding environmentally undesirable flaring or costly reinjection of the associated gas into the reservoir. Systems according to the present disclosure also offer advantages at offshore production facilities since hydrotreating of the reaction products may not be necessary, thus reducing the weight and footprint when compared with a conventional Fischer-Tropsch system including post-reactor wax hydrocracking and reducing the need for hydrogen associated with hydrocracking Furthermore, the liquid hydrocarbons produced by the present system may be blendable with crude oil produced from the well. In certain oilfields producing heavy oil, liquid hydrocarbons produced by the present system may be used as a diluent to improve pumpability of the crude oil produced from the well, thus lowering compression costs. In such embodiments, the heavy crude oil is diluted with a sufficient amount of liquid hydrocarbons produced by the present system to achieve a desired viscosity. By “heavy crude oil” is meant a crude oil having an API gravity of less than 20° as measured according to ASTM D287.

While various embodiments have been described, it is to be understood that it is within the scope of the skilled person to determine and select the most appropriate conditions for a specific reactor configuration and reaction regime. Such variations and modifications are to be considered within the purview and scope of the claims appended hereto.

What is claimed is:

1. A system for producing liquid hydrocarbons, the system comprising:
at least one reactor wherein a first synthesis gas feed, in the presence of a catalyst comprising a synthesis gas conversion component and an acidic component, is converted into an effluent comprising gaseous components, water and liquid hydrocarbons having a cloud point less than about 15° C. as determined by ASTM D 2500-09; and
a separator for separating the effluent into the gaseous components, the water and the liquid hydrocarbons;
wherein the effluent is provided to the separator without the removal of a solid wax phase.

2. The system of claim 1, wherein the at least one reactor includes at least two reactors arranged in series.

3. The system of claim 2, further comprising a means for introducing a hydrogen containing gas to a feed to the second reactor.

4. The system of claim 2, further comprising a pump for pumping the effluent from the first reactor to the second reactor; wherein the second reactor is at an elevated height relative to the first reactor.

5. The system of claim 1, further comprising a line which is not provided with a source of heat for removing effluent from the at least one reactor.

6. The system of claim 1, further comprising a valve which is not provided with a source of heat for controlling the flow of effluent from the at least one reactor.

7. The system of claim 1, further comprising a pump which is not provided with a source of heat for controlling the flow of effluent from the at least one reactor.

8. The system of claim 1, further comprising a reservoir for collecting the liquid hydrocarbons and a pump for recycling the collected liquid hydrocarbons to the at least one reactor.

9. The system of claim 1, wherein the system is located at one of a remote landlocked oil production facility and an offshore oil production facility.

10. A process for converting synthesis gas to a liquid hydrocarbon product comprising the following steps:
a. contacting a synthesis gas feed comprising hydrogen and carbon monoxide having a H₂/CO ratio between about 0.5 and about 2.5 with a catalyst comprising a synthesis gas conversion component and an acidic component in a reactor at a temperature between about 160° C. and about 350° C., a pressure between about 1 and about 100 atmospheres, and a gaseous hourly space velocity less than 20,000 volumes of gas per volume of catalyst per hour to produce an effluent; and
b. separating the effluent in a separator at a temperature of at least about 0° C. into a gaseous phase, an aqueous phase and a liquid hydrocarbon phase having a cloud point less than about 15° C. as determined by ASTM D 2500-09;
wherein the effluent is provided to the separator without the removal of a solid wax phase.

11. The process of claim 10, wherein the liquid hydrocarbon phase comprises:
i. 0 to 20 wt % CH₄;
ii. 0 to 30 wt % C₂-C₄;
iii. 50 to 95 wt % C₅₊; and
iv. 0 to 5 wt % C₂₁₊ normal paraffins.

12. The process of claim 10, wherein the liquid hydrocarbon phase has a Total Acid Number less than about 0.5.

13. The process of claim 10, wherein the aqueous phase has a Total Acid Number less than about 0.5.

14. The process of claim 10, wherein the catalyst comprises a synthesis gas conversion component and an acidic component disposed on integral particles.

15. The process of claim 10, wherein the reactor contains the synthesis gas conversion component in a catalyst bed upstream of the acidic component.

16. The process of claim 10, wherein the catalyst comprises a synthesis gas conversion component and an acidic component disposed on discrete particles which are mixed together.

17. The process of claim 10, further comprising diluting a heavy crude oil with the liquid hydrocarbon phase.

18. The process of claim 10, wherein the aqueous phase has a chemical oxygen demand of less than 300 mg/l.

19. The process of claim 10, further comprising:
d. adding hydrogen to the gaseous phase to form a second synthesis gas feed having a H₂/CO ratio between about 0.5 and about 2.5;
e. contacting the second synthesis gas feed with a second catalyst comprising a synthesis gas conversion component and an acidic component in a second reactor at a temperature between about 160° C. to about 350° C., a pressure between about 1 to about 100 atmospheres, and a gaseous hourly space velocity less than 20,000 volumes of gas per volume of catalyst per hour to produce a second effluent;
f. separating the second effluent into a second gaseous phase, a second aqueous phase and a second liquid hydrocarbon phase; and
g. combining the first and second liquid hydrocarbon phases to form a liquid hydrocarbon product having a cloud point less than about 15° C. as determined by ASTM D 2500-09.

20. The process of claim 19, wherein the CO conversion within each of the first and second reactors is between about 1 and about 80 mol % and the overall CO conversion of the process is between about 1 and about 99 mol %.

21. The process of claim 19, further comprising recycling the second vapor phase to at least one of the first and second reactors.

22. The process of claim 19, further comprising sending the first liquid hydrocarbon phase to the second reactor.

23. The process of claim 19, further comprising recycling the second liquid hydrocarbon phase to at least one of the first and second reactors.
